(19) United States
(12) Patent Application Publication
(10) Pub. No.: US 2026/0096889 A1
(43) Pub. Date: Apr. 9, 2026

(54) ARRANGEMENT OF IMPLANTABLE PRESSURE SENSOR IN AN ELECTRONIC IMPLANTABLE DEVICE

(71) Applicant: Boston Scientific Scimed, Inc., Maple Grove, MN (US)

(72) Inventors: Thomas Sinnott, Wexford (IE); Daragh Nolan, Youghal (IE); Brian P. Watschke, Minneapolis, MN (US); Richard Percy, Leamlara (IE)

(21) Appl. No.: 18/906,871

(22) Filed: Oct. 4, 2024

(51) Int. Cl. A61F 2/26; A61F 2/48; G01L 7/08

(52) U.S. Cl. CPC A61F 2/26; A61F 2/482; G01L 7/08; A61F 2210/0009; A61F 2220/0058

(57) ABSTRACT

A pressure sensor is configured to be positioned in a receptacle of a manifold of a fluid control system and in fluidic connection with fluid in a fluid passageway defined by the manifold. The pressure sensor includes: a metal housing including one or more interior cavities, the metal housing being configured to fit entirely within the receptacle; a flexible metal diaphragm attached to the metal housing and having a first portion positioned between an interior cavity of the one or more interior cavities and the fluid passageway and the first portion being configured to move inward and outward with respect to an interior cavity in response to a fluid pressure in the fluid passageway; and electrical circuitry configured for converting a pressure of fluid in the fluid passageway into an electrical signal.

### TECHNICAL FIELD

This disclosure relates generally to bodily implants, and more specifically to arrangements of pressure sensors in electronic implantable devices.

### BACKGROUND

Active implantable fluid operated inflatable devices can include one or more pumps that regulate a flow of fluid between different portions of the implantable device. One or more valves can be positioned within fluid passageways of the device to direct and control the flow of fluid to achieve inflation, deflation, pressurization, depressurization, activation, deactivation and the like of different fluid filled implant components of the device. In some implantable fluid operated devices, an implantable pumping device may be manually operated by the user to provide for the transfer of fluid between a reservoir and the fluid filled implant components of the device. Manipulation of the manually operated implantable pumping device may be challenging for some patients. Further, such manual operation of the pumping device may make it difficult to achieve consistent inflation, deflation, pressurization, depressurization, activation, deactivation and the like of the fluid filled implant components. Inconsistent inflation, deflation, pressurization, depressurization, activation and/or deactivation of the fluid filled implant device(s) may adversely affect patient comfort, efficacy of the device, and the overall patient experience. Some implantable fluid operated devices may include an electronic control system including an electronically controlled manifold providing for the transfer of fluid within the implantable fluid operated device. The use of the electronic control system may provide for more accurate actuation and control of the flow of fluid between components of the inflatable device, thus improving performance and efficacy of the device, as well as patient comfort and safety. In both a manually operated implantable pumping device and in an electronically controlled pumping device, one or more pressure sensors may be used to monitor pressures of fluids in different parts of the device. However, the inclusion of one or more pressure sensors in the pumping device can add bulk, weight, and complexity to the implantable device, where a goal for the implantable device may be for the device to be minimally invasive for a patient in whose body the device is implanted.

### SUMMARY

According to a general aspect, an implantable fluid operated device, includes: a fluid reservoir; an inflatable member; and a fluid control system configured to control fluid flow between the fluid reservoir and the inflatable member. The fluid control system includes: a manifold including a fluidic architecture defining one or more fluid passageways within in the manifold, with the manifold defining a plurality of receptacles, each receptacle configured for receiving a fluid control device; at least one pump positioned in a first receptacle of the receptacles and in fluidic connection with at least one of the one or more fluid passageways, the at least one pump being configured to pump fluid from the fluid reservoir to the inflatable member; a pressure sensor positioned in a second receptacle of the receptacles and in fluidic connection with one of the fluid passageway. The pressure sensor includes: a metal housing having one or more interior cavities, with the metal housing being configured to fit entirely within the second receptacle; electrical circuitry configured for converting a pressure into an electrical signal; a flexible metal diaphragm attached to the metal housing and having a first portion positioned between an interior cavity of the one or more interior cavities and a fluid passageway and the first portion being configured to move inward and outward with respect to an interior cavity in response to a fluid pressure in the fluid passageway.

Implementations can include one or more of the following features, alone, or in any combination with each other.

For example, the second receptacle can include a substantially circular shelf, and the metal housing of the pressure sensor can include a substantially circular flange seated on the substantially circular shelf.

For example, a depth of the substantially circular shelf below a top surface of the manifold can be greater than a thickness of the flange.

For example, the flange can be located at an end of the metal housing.

For example, the manifold can include metal, and the housing of the pressure sensor can be welded to the manifold.

For example, the implantable fluid operated device can further include a hermetically-sealed housing that contains the fluid control system, an electronic control system, and an energy storage device.

For example, the hermetically-sealed housing can include a first portion configured for receiving the fluid control system, the electronic control system, and the energy storage device and a second portion that is hermetically-sealed to the first portion.

For example, the pressure sensor can further include a printed circuit board, the printed circuit board including a plurality of electrical connectors configured for providing electrical signals generated by the pressure sensor for transmission to the electronic control system.

For example, the pressure sensor can include a receptacle within the metal housing, where the receptacle can be defined by an inner cylindrical wall of the metal housing and by the printed circuit board.

For example, the implantable fluid operated device can further include a plurality of flexible wires that electrically connect the plurality of electrical connectors to the electronic control system.

For example, the flexible wires can be electrically connected to the plurality of electrical connectors within the receptacle within the metal housing.

For example, the electronic control system can include an ASIC configured for processing electrical signals received from a MEMS sensor of the pressure sensor, where the pressure sensor in the fluid control systems does not include an ASIC.

For example, the electronic control system can include an ADC configured for processing electrical signals received from a MEMS sensor of the pressure sensor, where the pressure sensor in the fluid control systems does not include an ADC.

For example, the pressure sensor cannot extend out of the second receptacle above a top surface of the manifold.

In some aspects, the techniques described herein relate to a pressure sensor configured to be positioned in a receptacle of a manifold of a fluid control system and in fluidic connection with fluid in a fluid passageway defined by the manifold, the pressure sensor including: a metal housing including one or more interior cavities, the metal housing being configured to fit entirely within the receptacle; a flexible metal diaphragm attached to the metal housing and having a first portion positioned between an interior cavity of the one or more interior cavities and the fluid passageway and the first portion being configured to move inward and outward with respect to an interior cavity in response to a fluid pressure in the fluid passageway; and electrical circuitry configured for converting a pressure of fluid in the fluid passageway into an electrical signal.

Implementations can include one or more of the following features, alone, or in any combination with each other.

For example, the metal housing of the pressure sensor can include a substantially circular flange configured to seat on a substantially circular shelf within the receptacle of the manifold.

Implementations can include one or more of the following features, alone, or in any combination with each other.

For example, a depth of the substantially circular shelf below a top surface of the manifold can be greater than a thickness of the flange and the flange can be located at an end of the metal housing.

For example, the metal housing can include titanium.

For example, the pressure sensor in the fluid control systems may not include an ASIC configured for processing electrical signals received from a MEMS sensor of the pressure sensor.

For example, the pressure sensor in the fluid control systems may not include an ADC configured for processing electrical signals received from a MEMS sensor of the pressure sensor.

### BRIEF DESCRIPTION OF THE DRAWINGS

FIG. 1 is a block diagram of an implantable fluid operated inflatable device according to an aspect.

FIG. 2 illustrates a system including an example implantable fluid operated inflatable device according to an aspect.

FIG. 3 is a schematic diagram of a fluidic architecture of an implantable fluid operated inflatable device according to an aspect.

FIG. 4A is a schematic cutaway perspective view of an example pressure sensor.

FIG. 4B is a cross-sectional view of the example pressure sensor of FIG. 4A.

FIG. 5 is a schematic perspective view of a manifold that can be used in a fluid control system of an implantable device.

FIG. 6A is a schematic top view of system including a manifold, an electronic control system, and an energy storage device contained within a first portion of a hermetic housing.

FIG. 6B is a schematic section view of the system through section A-A′ of FIG. 6A.

FIG. 6C is a schematic section view of the system through section B-B′ of FIG. 6A.

FIG. 7A is a schematic cutaway perspective view of another example pressure sensor.

FIG. 7B is a cross-sectional view of the example pressure sensor of FIG. 7A.

FIG. 8A is a schematic top view of system including a manifold, an electronic control system, and an energy storage device contained within a first portion of a hermetic housing.

FIG. 8B is a schematic section view of the system through section A-A′ of FIG. 8A.

FIG. 8C is a schematic section view of the system through section B-B′ of FIG. 8A.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

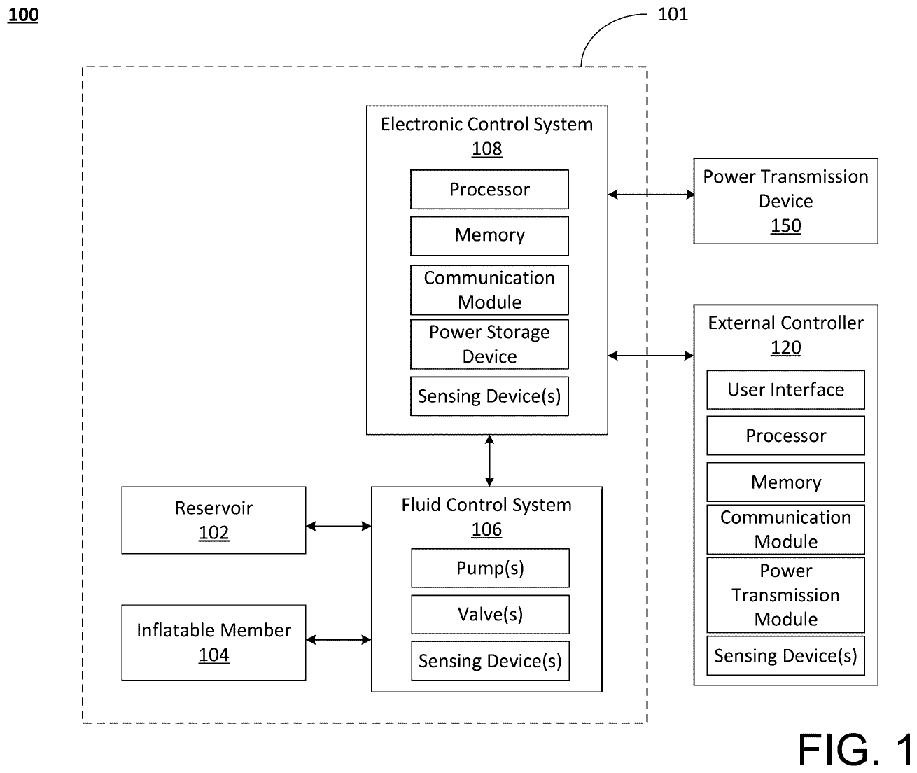

FIG. 1 is a block diagram of an example implantable fluid operated inflatable device 100. The example inflatable device 100 shown in FIG. 1 includes implantable components 101, including a fluid reservoir 102, a fluid receiver (e.g., an inflatable member) 104, a fluid control system 106, and an electronic control system 108. The electronic control system 108 may interface with the fluid control system 106, and the fluid control system 106 can include fluidics components such as one or more pumps, one or more valves and the like configured to transfer fluid between the fluid reservoir 102 and the fluid receiver 104. The fluid control system 106 can include one or more sensing devices (e.g., pressure sensors, flow rate sensors, thermometers, etc.) that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid operated inflatable device 100 and/or communication with one or more external device(s). In some examples, the electronic control system 108 includes components such as a processor, a memory, a communication module, a power storage device, or battery, sensing devices such as, for example, an accelerometer, and other such components configured to provide for the operation and control of the implantable fluid operated inflatable device 100. In some examples, the communication module of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in, or incorporated into, a single housing. In some implementations, at least a portion of the electronic control system 108 is physically separate from the fluid control system 106. In some implementations, components of the fluid control system 106 can be included in an integrated manifold. In some implementations, some modules of the electronic control system 108 are coupled to, or incorporated into, the fluid control system 106, and some modules of the electronic control system 108 are separate from the fluid control system 106. For example, in some implementations, different components of a pressure sensor can be included in the fluid control system 106 and in the electronic control system 108, where the fluid control system 106 and the electronic control system 108 are physically separate but the different components of the pressure communicate between their locations in the fluid control system 106 and the electronic control system 108 to provide the functionality of the pressure sensor. For example, the pressure sensor can include a die, a bridge circuit, (e.g., a Wheatstone bridge), an amplifier, a signal convertor, a compensation or calibration circuit, and a PCBA (printed circuit board assembly), and some of these components can be included in the electronic control system 108, rather than in the fluid control system 106.

In some examples, electronic monitoring and control of the fluid operated inflatable device 100 may provide for improved patient control of the device, improved patient comfort, improved patient safety, and the like. In some examples, electronic monitoring and control of the fluid operated device 100 may afford the opportunity for tailoring of the operation of the inflatable device 100 by a physician without further surgical intervention. Fluidic architecture defining the flow and control of fluid through the fluid operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

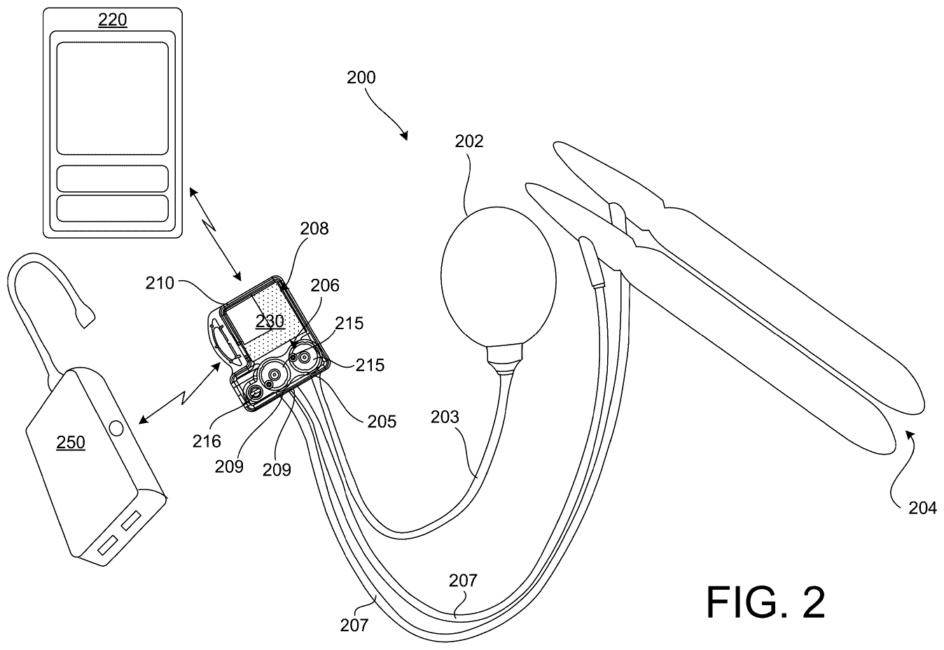

The example implantable fluid operated inflatable device 100 may be representative of a number of different types of implantable fluid operated devices. For example, the device 100 shown in FIG. 1 may be representative of an inflatable penile prosthesis as shown in FIG. 2. In some implementations, the example implantable fluid operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid operated inflatable device 200 in the form of an example inflatable penile prosthesis is shown in FIG. 2. The example inflatable device 200 includes a fluid reservoir 202, one or more inflatable members 204, a fluid control system 206 and an electronic control system 208 contained within a housing 210, a conduit 203 that connects the reservoir to the housing fluid control system 206, and conduits 207 that connect the inflatable members 204 to the fluid control system. The fluid control system 206, which can be similar to the example fluid control system 106 described above with respect to FIG. 1, includes fluidics components such as pumps, valves, sensing devices and the like positioned in fluid passageways of a manifold. In some implementations, the fluid control system includes one or more fluid control devices 215 (e.g., pumps, values, or combined pump/valves), one or more pressure sensors 216, and other such components. The electronic control system 208, which can be similar to the example electronic control system 108 described above with respect to FIG. 1, is configured to provide for the transfer of fluid between a reservoir 202 (such as the example reservoir 102 described above with respect to FIG. 1) and an inflatable member 204 (similar to the example inflatable member or inflatable member 104 described above with respect to FIG. 1) in the form of a pair of inflatable cylinders, via the fluidics components. Fluidics components of the fluid control system 206, and electronic components of the electronic control system 208 may be received in a housing 210. In FIG. 2, a transparent view of the housing 210 is shown to illustrate components contained within the housing, such as the fluid control system 206, the electronic control system 208.

In some implementations, fluidics components of the fluid control system 206 received in the housing 210 may define a fluid manifold 230 that provides for the control of the flow of fluid between the reservoir 202 and the inflatable member 204. A first conduit 203 connects a first fluid port 205 of the fluid manifold 230 with the reservoir 202. One or more second conduits 207 connect one or more second fluid ports 209 of the fluid manifold 230 with the inflatable member 204 in the form of the inflatable cylinders. The electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 may be charged and/or recharged by a power transmission module of the external controller 220, and/or by a power transmission device 250, that is separate from the external controller 220.

The principles to be described herein may be applied to the example implantable fluid operated inflatable device, in the form of the inflatable penile prostheses shown in FIG. 2, and other types of implantable fluid operated inflatable devices that rely on a pump assembly including various fluidics components to provide for the transfer of fluid between the different fluid filled implantable components to achieve inflation, deflation, pressurization, depressurization, deactivation, occlusion, and the like for effective operation. The example implantable fluid operated inflatable device 200 shown in FIG. 2 includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. However, in some situations, a size and/or a configuration of the electronic control system 208 and/or the fluid control system 206 may pose a challenge for some patients. Accordingly, in some implementations, the fluid manifold 230 may include a fluid control system 206 having one or more combined pump and valve devices and may include a pressure sensor integrated into a manifold of the fluid control system 206 and arranged to occupy a small volume within the housing 210. The use of combined pump and valve devices and the integrated pressure sensor may reduce a number of active components within the fluid manifold 230, thus reducing the overall size of the fluid manifold 230.

A fluid control system, in accordance with implementations described herein, can include a pump assembly including, for example, one or more pump and valve devices within a fluid circuit of the pump assembly to control the transfer fluid between the fluid reservoir and the inflatable member. In some examples, the pump assembly including the one or more pump and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump/valve device(s) include piezoelectric elements. In some examples, the pump assembly includes one or more pressure sensing devices in the fluid circuit to provide for precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid operated device to provide for patient safety and device efficacy.

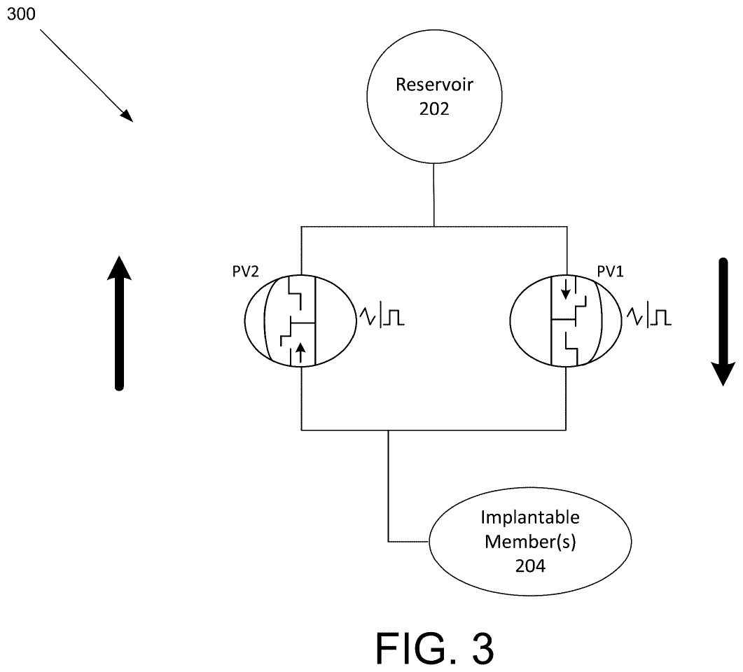

FIG. 3 is a schematic diagram of an example fluidic architecture for an implantable fluid operated inflatable device, according to an aspect. The fluidic architecture shown in FIG. 3 includes combination pump/valves positioned between the reservoir 202 and the inflatable member 204, to control the flow of fluid between the reservoir 202 and the inflatable member 204. The fluidic architecture of an implantable fluid operated inflatable device can include other arrangements of fluidic channels, pump(s)/valve(s), pressure sensor(s) and other components than shown in FIG. 3.

In particular, the example fluidic architecture 300 shown in FIG. 3 includes a first fluid control device, or combined pump and valve device, PV1 positioned in a first fluid passageway and controlling the flow of fluid from the reservoir 202 to the inflatable member 204, and a second fluid control device, or combined pump and valve device, PV2 positioned in a second fluid passageway and controlling the flow of fluid from the inflatable member 204 to the reservoir 202. The first and second fluid passageways can be conduits through which fluid flows within the fluidic architecture 300 between the reservoir 202 and the inflatable member(s) 204.

In the example arrangement shown in FIG. 3, the first combined pump and valve device PV1 and the second combined pump and valve device PV2 may be operated in a first mode to inflate or pressurize the inflatable member 204, and in a second mode to deflate or repressurize the inflatable member 204. In the first mode of operation, the first combined pump and valve device PV1 may be operable to convey fluid from the reservoir 202 to the inflatable member 204, while the second combined pump and valve device PV2 remains closed/inoperable to prevent flow of fluid from the inflatable member 204 towards the reservoir 202 to prevent deflation/depressurization. The first combined pump and valve device PV1 may remain operable to pump fluid to the inflatable member 204 until a desired pressure is achieved. The first combined pump and valve device PV1 may be closed once the desired pressure is achieved, to maintain the inflatable member 204 at the desired pressure/inflated state. In the second mode of operation, the second combined pump and valve device PV2 may be operable to convey fluid from the inflatable member 204 to the reservoir 202, while the first combined pump and valve device PV1 remains closed/inoperable to prevent flow of fluid from the reservoir 202 towards the inflatable member 204 to prevent inflation/pressurization. The second combined pump and valve device PV2 may remain operable to pump fluid to the reservoir 202 until a desired pressure is achieved at the inflatable member 204. The second combined pump and valve device PV2 may be closed once the desired pressure is achieved, to maintain the inflatable member 204 at the desired pressure/in the deflated state.

In the example implantable fluid operated devices described herein, a pressure sensor can be included in the device to monitor and/or measure one or more pressures of fluid in the devices. An electrical signal from the pressure sensor can then be used to control the pressure of the fluid in the device, for example, to optimize a performance of the device or to prevent damage to the device or to a user in whom the devices implanted.

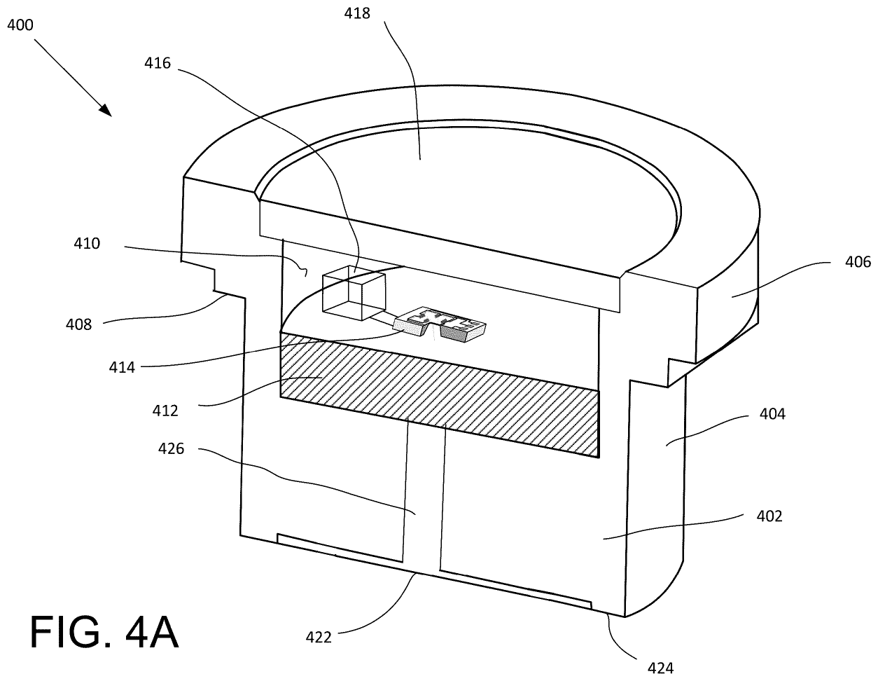

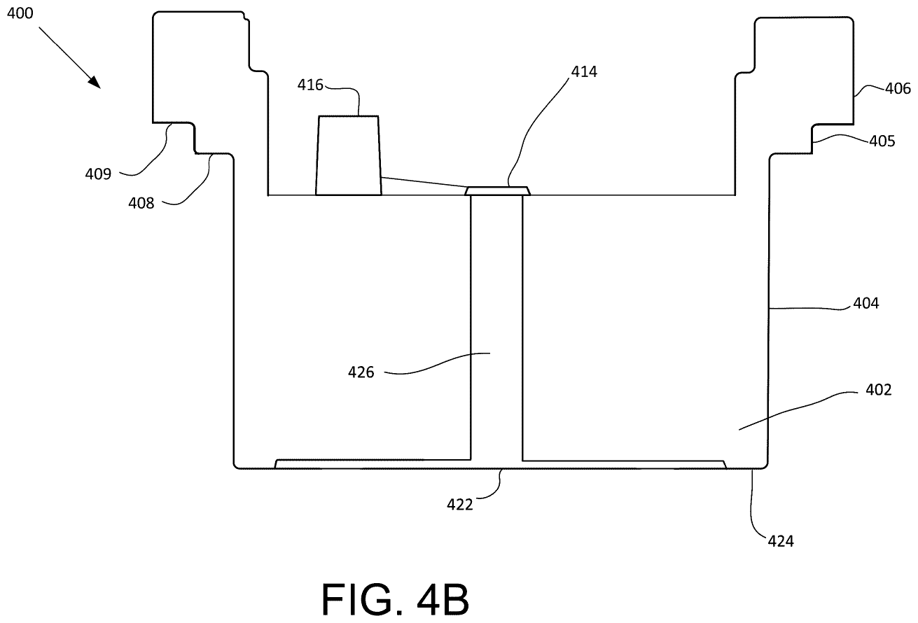

FIG. 4A is a schematic cutaway perspective view of an example pressure sensor 400, and FIG. 4B is a cross-sectional view of the example pressure sensor 400. The pressure sensor can include a metal housing 402, which, in some implementations, can have a generally cylindrical shape, with one or more circular sidewalls 404, 405, 406, which can have different diameters. In some implementations, the metal housing 402 can be made of titanium or a titanium alloy. In an implementation in which the sidewalls 404, 405, 406 have different diameters, the metal housing 402 can include a first flange 408 that extends radially outward from a diameter of a first sidewall 404. The first flange 408 can engage with a substantially circular shelf in a receptacle or recess of the manifold so that the pressure sensor 400 can fit into the receptacle or recess of the manifold of an implantable fluid operated device, with the first flange 408 being mechanically coupled to, or seated on, the corresponding substantially circular shelf of the manifold of the implantable fluid operated device. The housing 402 of the pressure sensor 400 can include a second flange 409 that extends radially inward from the second sidewall 406, and the second flange 409 can engage with a top surface of the manifold, where the top surface defines an end of the recess or receptacle in which the pressure sensor fits, so that the sidewall 406 is above, and not within the recess or receptacle of the manifold.

The metal housing 402 can define one or more interior cavities within the housing. For example, the metal housing can include an upper cavity 410 that is configured at least for holding electronic components of the pressure sensor. The upper cavity 410 can house a printed circuit board 412 on which electrical circuitry and/or electrical components are connected. For example, the electrical circuitry can include, among other things, a sensor (e.g., a MEMS sensor) 414 and an application specific integrated circuit (ASIC) 416 that are connected to the printed circuit board 412. The ASIC 416 can receive electrical signals from the sensor 414 and process the signals before sending the processed signals to the processor that is part of an electronic control system of an implantable device.

The pressure sensor 400 can include a top plate 418 that can be fitted onto the metal housing 402 to close the upper cavity 410 after the electrical circuitry is positioned within the upper cavity 410. The top plate 418 can be used to locate and retain electrical connectors that electrically connect components within the housing 402 to components outside the housing. In some implementations, the top plate 418 can hermetically seal against the housing 402, so liquid cannot enter the interior of the housing 402 between the top plate 418 and the housing 402. In some implementations, the top plate 418 can be glued, welded, or otherwise attached to the housing 402. In some implementations, the top plate can be sealed against the housing 402 with a connection that does not rely on a welded joint between the top plate 418 and the housing 402. For example, a flexible O-ring between the top plate 418 and the housing 402 can form the hermetic seal between the top plate 418 and the housing 402. The pressure sensor 400 can include one or more electrical connectors (not shown) that extend through the top plate 418 to receive electrical signals from, and to provide electrical signals to, the electrical circuitry housed within the upper cavity 410 of the pressure sensor.

The pressure sensor 400 also can include a flexible metal diaphragm 422 that is attached to a bottom portion of the metal housing 402. The flexible metal diaphragm 422 can be made from the same material as the metal housing 402, such as, for example, titanium or titanium alloy and can have a small thickness of, for example, 40 μm or less, 25 μm or less, or 16 μm or less.

In an implementation in which the metal housing 402 includes a cylindrical sidewall 404, the metal housing 402 can include a bottom perimeter rim 424 at a bottom of the cylindrical sidewall 404, and the flexible metal diaphragm 422 can be attached to the bottom perimeter rim. The metal housing 402 of the pressure sensor 400 can additionally define an interior cavity 426 that can be filled with a fluid (e.g., an incompressible silicone oil). When the flexible metal diaphragm is attached to the metal housing 402, fluid in the interior cavity 426 can mechanically and fluidically couple movement of the flexible metal diaphragm 422 to the MEMS sensor 414 on the printed circuit board 412. In this manner, when the pressure sensor 400, or at least a lower portion of the housing 402 and the metal diaphragm 422, is placed into fluid connection with fluid in a fluid passageway of a fluidic system, a pressure of fluid in the fluid passageway and outside the pressure sensor 400 on the flexible metal diaphragm 422 can be transmitted to the MEMS sensor 414. For example, after the interior cavity 426 is filled with fluid, with the flexible metal diaphragm attached to the metal housing 402, electrical signals due to pressure of the fluid in the cavity 426 on the MEMS sensor 414 can be calibrated against known pressures outside the housing 402. Then, variations in pressure of fluid on an outside surface of the flexible metal diaphragm 422 can cause the diaphragm to flex and move toward or away from the MEMS sensor 414 and, because the fluid in the cavity 426 has a low compressibility, the movement of the diaphragm 422 results in movement of a corresponding mechanical element of the MEMS sensor 414, which is converted to an electrical signal representing a pressure on the outside of the flexible metal diaphragm 422.

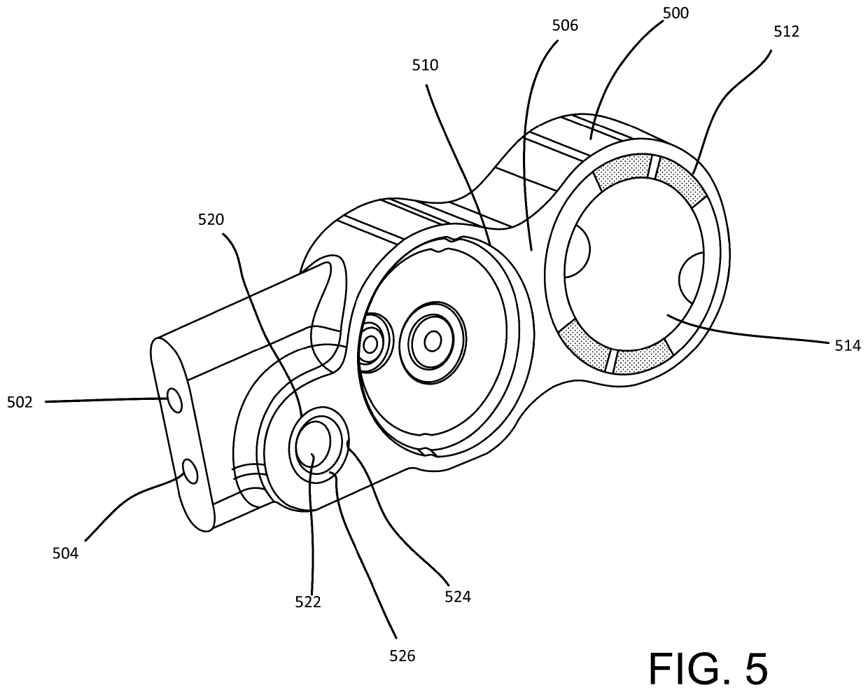

FIG. 5 is a schematic perspective view of the manifold 500 that can be used in a fluid control system of an implantable device, such as, for example, the inflatable device 200. The manifold 500 can include a first port 502 that can be fluidically connected to the fluid reservoir and a second port 504 that can be fluidically connected to one or more inflatable members. The manifold 500 can be configured to receive components of a fluid control system that can be used to control the flow of fluid in the implantable device, for example, to control the flow of fluid between the reservoir and the inflatable members.

In some implementations, the manifold 500 can include a first receptacle 510 that is configured for receiving a first fluid control device (e.g., a combined pump and valve device) that is adapted for controlling the flow of fluid in the implantable device. The first receptacle 510 can define an open cavity that extends below the top surface 506 of the manifold 500 into the body of the manifold 500. The manifold 500 can include a second receptacle 512 that is configured for receiving a second fluid control device (e.g., a combined pump and valve device) that is adapted for further controlling the flow of fluid in the implantable device. The first receptacle 510 is shown without a fluid control device inserted into the first receptacle 510, and the second receptacle 512 is shown with a fluid control device 514 inserted into the second receptacle 512. The fluid control device 514 in the second receptacle 512 and another fluid control device that could be inserted into the first receptacle 510 can be electrically actuated devices (e.g., which may include a piezoelectric element) and can be electronically controlled to pump fluid through ports 502, 504 between the reservoir and one or more inflatable members.

The manifold 500 can include a third receptacle 520 that is configured for receiving a sensing device, for example, a pressure sensor that is configured for measuring the pressure of fluid in a fluid pathway within the manifold 500. The third receptacle 520 can include a first circular well 522 that extends into the body of the manifold 500 to a first step below the top surface 506 of the manifold. The third receptacle 520 also can include a second circular well 524 having a central axis that is substantially identical to the central axis of the first circular well 522, where the second circular well 524 extends into the body of the manifold 500 to a second depth below the top surface 506 that is less than the first depth. The diameter of the second circular well 524 can be greater than the diameter of the first circular well 522, such that a substantially circular shelf 526 is defined within the third receptacle 520 at the second depth below the top surface 506 of the manifold 500.

The dimensions of the receptacle 520 can correspond to the dimensions of the sidewalls 404, 405, 406 and the flanges 408, 409 of the pressure sensor 400 of FIGS. 4A and 4B, so that the pressure sensor 400 can be received in the third receptacle with the flange 408 being coupled to the substantially circular shelf 526 and the flange 409 being outside the receptacle 520 and being above, supported by, or attached to, the top surface 506 of the manifold. Thus, the portion of the pressure sensor 400 that includes the sidewall 406 and any part of the pressure sensor located radially inward from the sidewall 406 is located outside of the receptacle 520 and above the top surface 506 of the manifold 500.

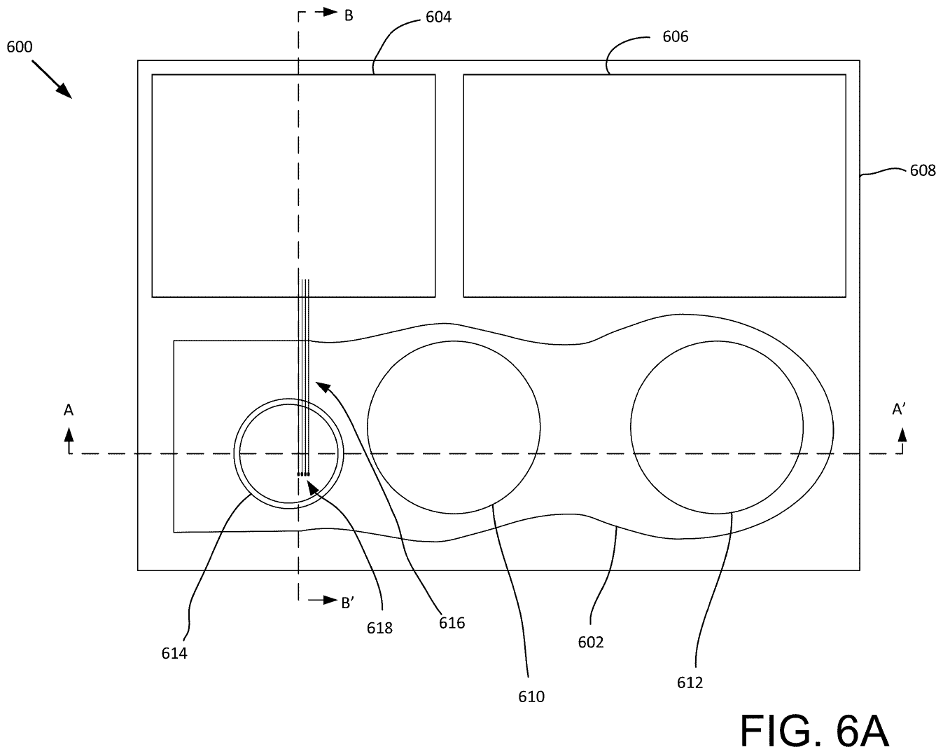

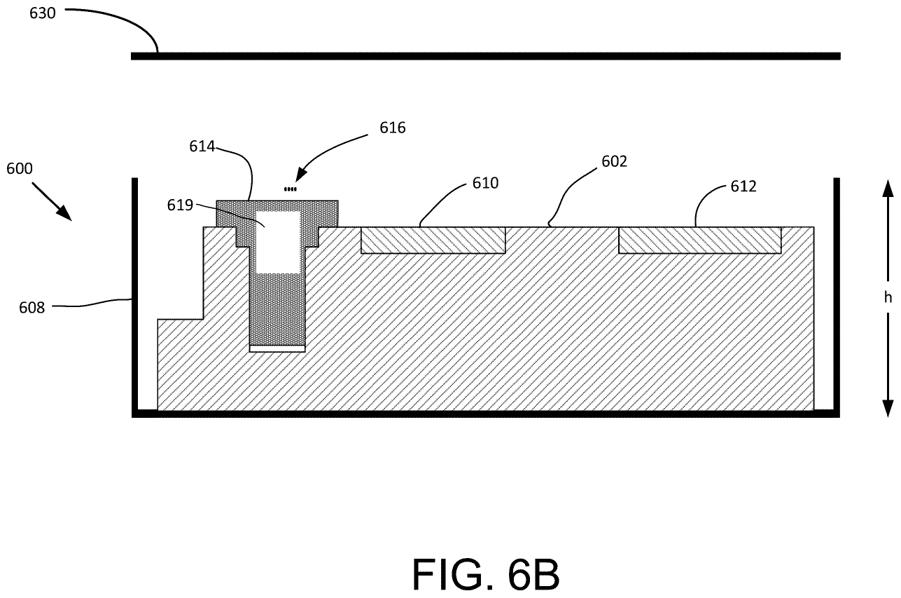

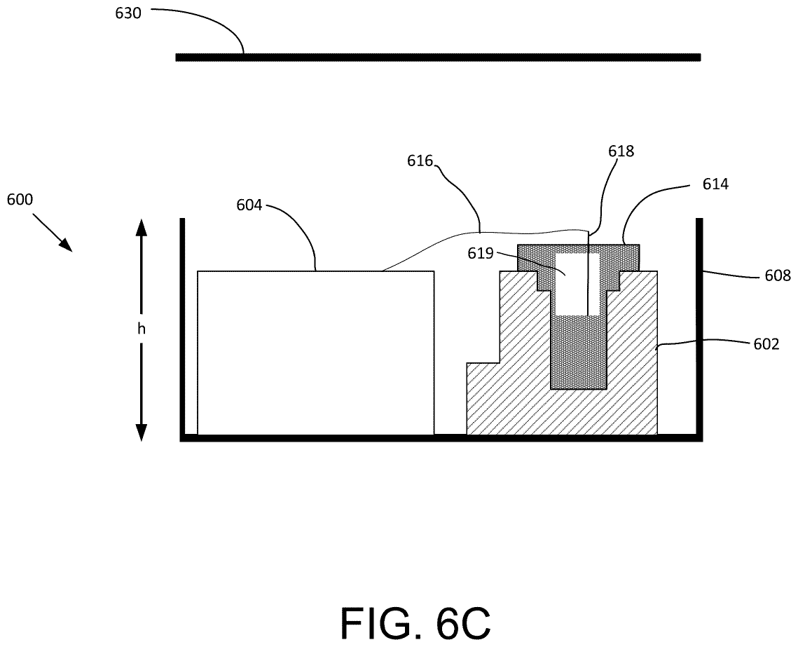

FIG. 6A is a schematic top view of system 600 including a manifold 602, an electronic control system 604, and an energy storage device 606 contained within a first portion of a hermetic housing 608. FIG. 6B is a schematic section view of the system 600 through section A-A′ of FIG. 6A. FIG. 6B is another schematic section view of the system 600 through section B-B′ of FIG. 6A. The manifold 602 can be configured to receive fluid control devices such as, for example, first and second combined fluid pump and valve devices 610, 612 and a pressure sensor 614. The electronic control system 604 can include electrical components, for example, a printed circuit board, a processor, memory, and other such components that are configured for controlling the operation of the first and second combined fluid pump and valve devices 610, 612 and for receiving information from the pressure sensor 614.

Electrical signals can be exchanged between the electronic control system 604 and the fluid control devices 610, 612 and between the electronic control system 604 and the pressure sensor 614. The electrical signals can be exchanged by way of one or more electrical connectors electrically connected between the electronic control system 604 and the devices 610, 612, 614. Electrical connectors 616 and 618 connected between the electronic control system 604 and the pressure sensor are shown, but electrical connectors between the electronic control system 604 and the devices 610, 612 are omitted for clarity. In the top view of FIG. 6A, a top plate of the pressure sensor is evident, with a plurality of electrical connectors 618 extending upward from a printed circuit board at a bottom of a cavity 619, which is defined by a housing and top plate of the pressure sensor, and protruding through the top plate of the pressure sensor 614.

As shown in FIG. 6A, one or more electrical connectors can extend out of the pressure sensor 614 (e.g., out through a top plate similar to top plate 418 shown in FIG. 4A). In addition, one or more wires 616 can be connected between the connectors 618 of the pressure sensor 614 and the electronic control system 604. In some implementations, the wires 616 can be flexible wires, and in some implementations that include multiple wires 616, the wires can be grouped or bound together in a flexible ribbon cable.

In FIGS. 6B and 6C, a second portion of the hermetic housing 630 is shown in addition to the system 600 that includes the manifold 602, the electronic control system 604, and the energy storage device 606 contained within the first portion of a hermetic housing 608. The manifold 602, the electronic control system 604, and the energy storage device 606 can be attached (e.g., adhesively bonded, welded, soldered, mechanically coupled, etc.) to the first portion of the hermitic housing 608, and then, after electrical connections are established between the electronic control system 604 and the pressure sensor 614 and between the electronic control system 604 and the fluid control devices 610, 612, the second portion of the hermetic housing 630 can be hermetically sealed to the first portion of the hermetic housing 608 to hermetically seal components 604, 606, 602, 610, 612, and 614 within an enclosed cavity defined by the portions 608, 630 of the hermetic housing. In some implementations, the first and second portions 608, 630 of the hermetic housing can include metal (e.g., titanium), and the second portion of the hermetic housing 630 can be hermetically sealed to the first portion of the hermetic housing 608 by welding the second portion to a top perimeter edge of the first portion. A height, h, of the cavity must be sufficient for the components of the system 600 to fit within the enclosed cavity defined by the portions 608, 630 of the hermetic housing. Although the enclosed cavity is shown as being formed from a rectilinear-shaped first portion of the hermetic housing 608 and a flat second portion of the hermetic housing 630, other shapes of the different portions of the hermetic housing also are possible.

To reduce the volume of the enclosed cavity needed to accommodate the components of the system that are housed in a hermetically-sealed cavity for implantation into a patient, the configuration of the pressure sensor can be adapted for use within the hermetically-sealed cavity. For example, because the pressure sensor is to be used in a hermetically-sealed cavity, components of the pressure sensor do not, themselves, need to be enclosed in a cavity to secure them from damage, and connectors that connect the pressure sensor to an electronic control system can be configured to help reduce a volume of the implantable hermetically-sealed cavity.

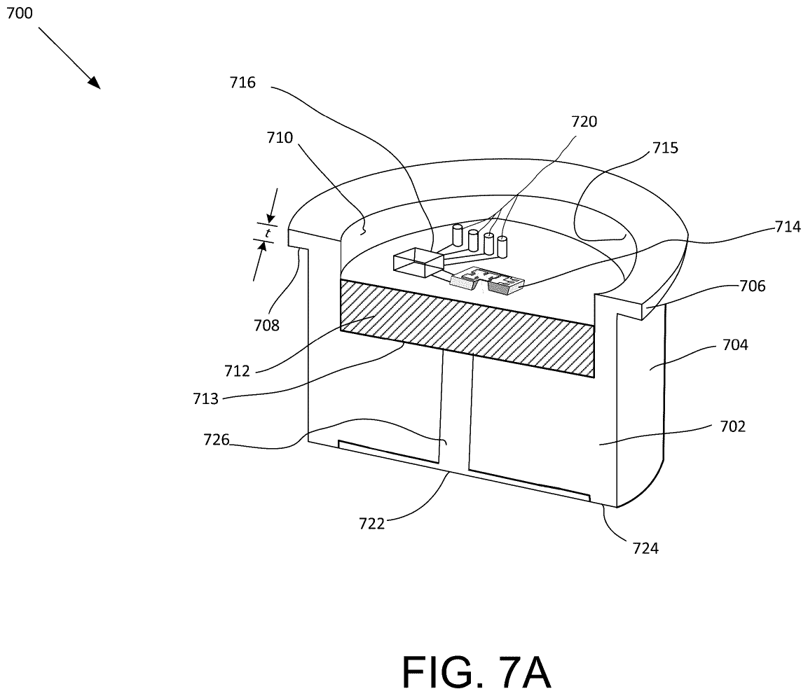

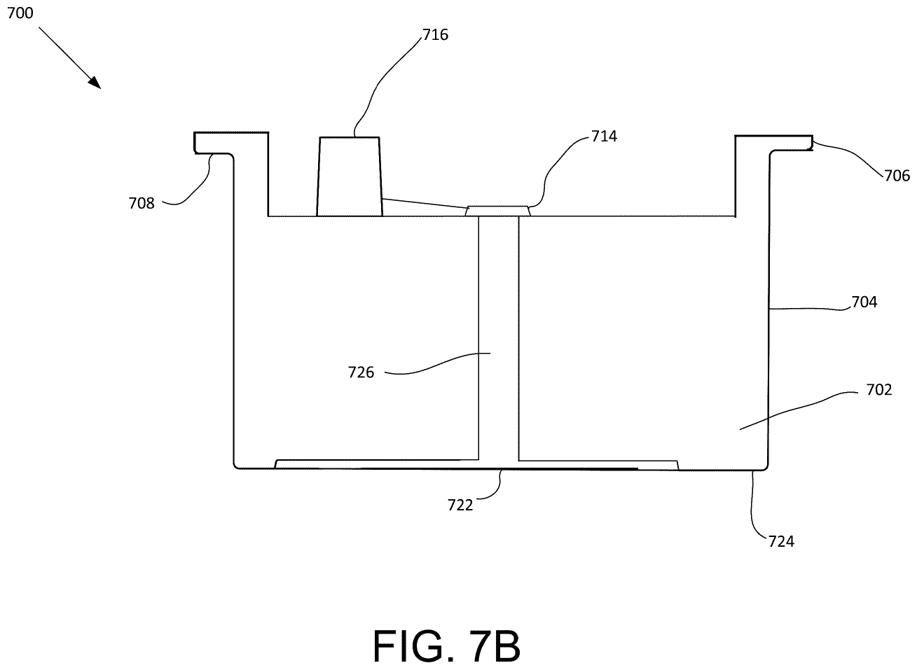

FIG. 7A is a schematic cutaway perspective view of another example pressure sensor 700, and FIG. 7B is a cross-sectional view of the example pressure sensor 700, where the pressure sensor is adapted for use within a hermetically-sealed cavity. In some implementations, the pressure sensor 700 includes an open receptacle in which electrical components can be disposed without being enclosed in a cavity and electrical connections between the electrical components of the pressure sensor and an electrical control system of an implantable device can be made without the connections passing through an exterior housing wall of the pressure sensor.

The pressure sensor 700 can include a metal housing 702, which, in some implementations, can have a generally cylindrical shape, with one or more circular sidewalls 704, 706, which can have different diameters. In some implementations, the metal housing 702 can be made of titanium or a titanium alloy. In an implementation in which the sidewalls 704, 706 have different diameters, the metal housing 702 can include a flange 708 that extends radially outward from a diameter of a first sidewall 704. The flange 708 can engage with the substantially circular shelf 526 in the receptacle or recess 520 of the manifold 500 so that the pressure sensor 700 can fit into the receptacle or recess of the manifold of an implantable fluid operated device, with the flange 708 mechanically coupled to, or seated on, the corresponding substantially circular shelf of the manifold of the implantable fluid operated device. A thickness, t, of the flange 708 can be less than a second depth of the substantially circular shelf 526 below the top surface 506 of the manifold 500, so that when the flange 708 seats on the substantially circular shelf, the flange does not protrude above the top surface 506 of the manifold 500, and the entirety of the pressure sensor housing 702 is located below the top surface 506 of the manifold.

The metal housing 702 can define one or more interior cavities and receptacles within the housing. For example, the metal housing can include an upper receptacle 710 that is configured at least for holding electronic components of the pressure sensor. The upper receptacle 710 can be defined, at least in part, by a portion 713 of the housing on which the printed circuit board 712 is disposed and the upper edge of the inner cylindrical wall 715 of the housing. The upper receptacle 710 can house a printed circuit board 712 on which electrical circuitry and/or electrical components are connected. For example, the electrical circuitry can include, among other things, a sensor (e.g., a MEMS sensor) 714 and an application specific integrated circuit (ASIC) 716 that are connected to the printed circuit board 712. The ASIC 716 can receive electrical signals from the sensor 714 and process the signals before sending the processed signals to the processor that is part of an electronic control system of an implantable device.

In contrast to the pressure sensor 400 of FIGS. 4A and 4B, the pressure sensor 700 need not include a top plate that fits onto the metal housing 702 to close the upper receptacle 710, although, in some implementations, the pressure sensor 700 may include such a top plate. The pressure sensor 700 can include one or more electrical connectors 720 that can be electrically connected to the printed circuit board 712 and through which electrical signals can be transmitted between electrical components of the pressure sensor (e.g., the ASIC 716) and electrical components that are external to the pressure sensor.

The pressure sensor 700 also can include a flexible metal diaphragm 722 that is attached to a bottom portion of the metal housing 702. The flexible metal diaphragm 722 can be made from the same material as the metal housing 702, such as, for example, titanium or titanium alloy and can have a small thickness of, for example, 40 μm or less, 25 μm or less, or 16 μm or less.

In an implementation in which the metal housing 702 includes a cylindrical sidewall 704, the metal housing 702 can include a bottom perimeter rim 724 at a bottom of the cylindrical sidewall 704, and the flexible metal diaphragm 722 can be attached to the bottom perimeter rim. The metal housing 702 of the pressure sensor 700 can additionally define an interior cavity 726 that can be filled with a fluid (e.g., an incompressible silicone oil). When the flexible metal diaphragm is attached to the metal housing 702, fluid in the interior cavity 726 can mechanically and fluidically couple movement of the flexible metal diaphragm 722 to the MEMS sensor 714 on the printed circuit board 712. In this manner, when the pressure sensor 700, or at least a lower portion of the housing 702 and the metal diaphragm 722, is placed into fluid connection with fluid in a fluid passageway of a fluidic system, a pressure of fluid in the fluid passageway and outside the pressure sensor 700 on the flexible metal diaphragm 722 can be transmitted to the MEMS sensor 714. For example, after the interior cavity 726 is filled with fluid, with the flexible metal diaphragm attached to the metal housing 702, electrical signals due to pressure of the fluid in the cavity 726 on the MEMS sensor 714 can be calibrated against known pressures outside the housing 702. Then, variations in pressure of fluid on an outside surface of the flexible metal diaphragm 722 can cause the diaphragm to flex and move toward or away from the MEMS sensor 714 and, because the fluid in the cavity 726 has a low compressibility, the movement of the diaphragm 722 results in movement of a corresponding mechanical element of the MEMS sensor 714, which is converted to an electrical signal representing a pressure on the outside of the flexible metal diaphragm 722.

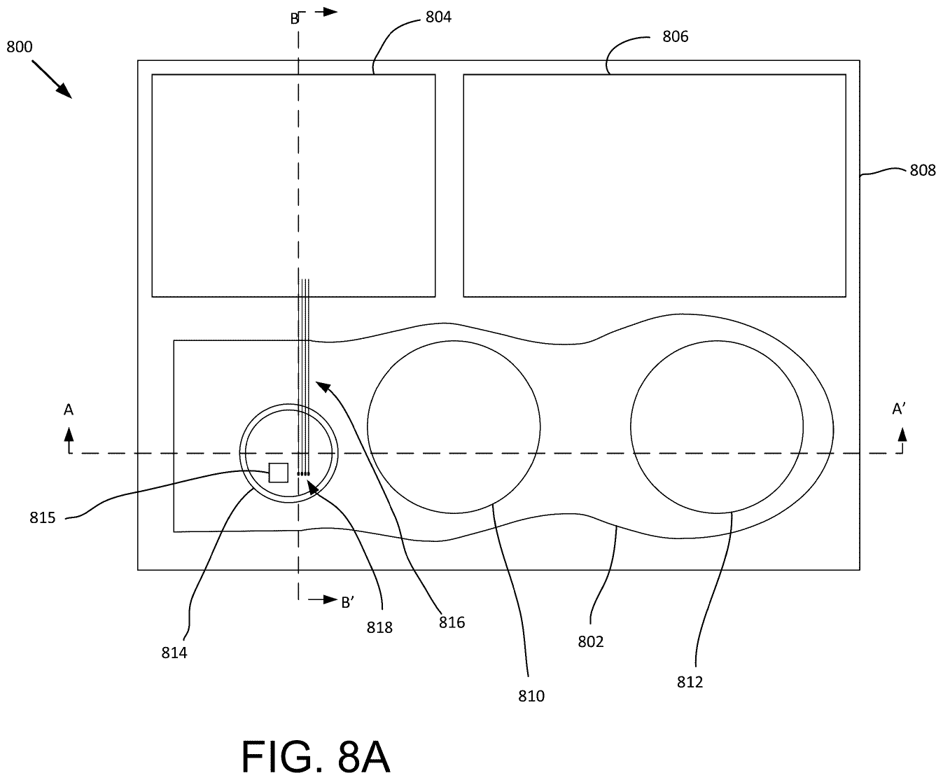

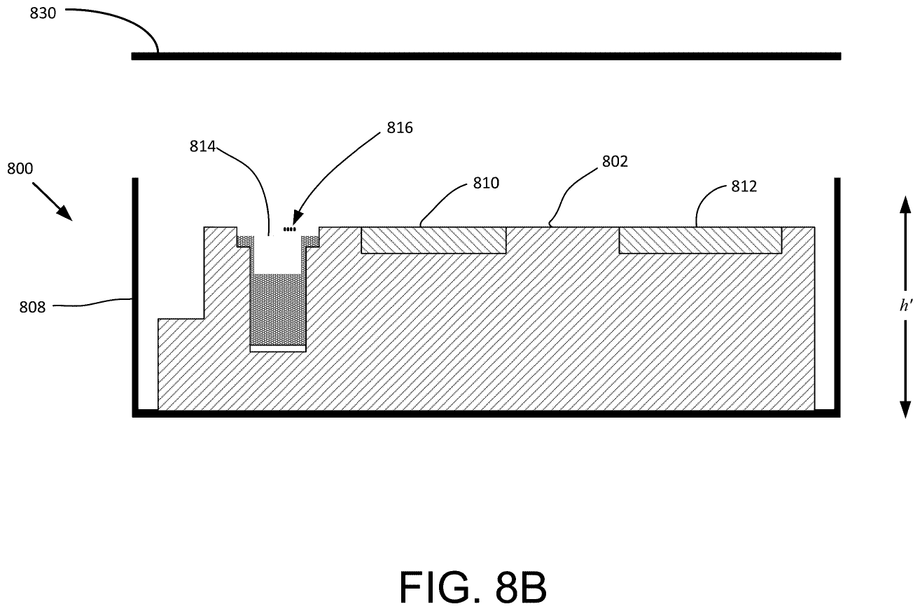

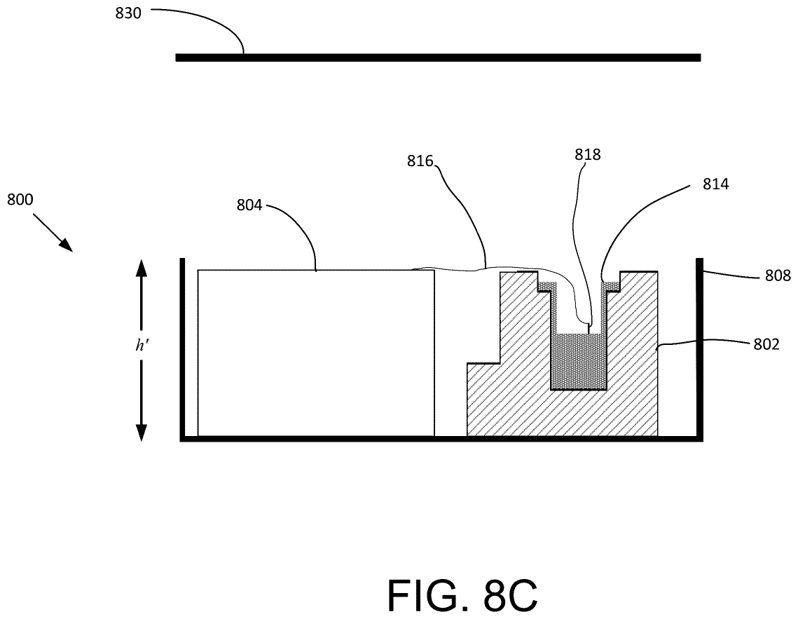

FIG. 8A is a schematic top view of system 800 including a manifold 802, an electronic control system 804, and an energy storage device 806 contained within a first portion of a hermetic housing 808. FIG. 8B is a schematic section view of the system 800 through section A-A′ of FIG. 8A. FIG. 8B is another schematic section view of the system 800 through section B-B′ of FIG. 8A. The manifold 802 can be configured to receive fluid control devices such as, for example, first and second combined fluid pump and valve devices 810, 812 and a pressure sensor 814. The electronic control system 804 can include electrical components, for example, a printed circuit board, a processor, memory, and other such components that are configured for controlling the operation of the first and second combined fluid pump and valve devices 810, 812 and for receiving information from the pressure sensor 814.

Electrical signals can be exchanged between the electronic control system 804 and the fluid control devices 810, 812 and between the electronic control system 804 and the pressure sensor 814. The electrical signals can be exchanged by way of one or more electrical connectors electrically connected between the electronic control system 804 and the devices 810, 812, 814. Electrical connectors 816 and 818 connected between the electronic control system 804 and the pressure sensor are shown, but electrical connectors between the system 804 and the devices 810, 812 are omitted for clarity. In the top view of FIG. 8A, a printed circuit board of the pressure sensor is evident, with an ASIC 815 disposed on the printed circuit board and with a plurality of electrical connectors 818 protruding from the printed circuit board. The pressure sensor 814 shown in FIG. 8A does not include a top plate, such as the top plate 418 of pressure sensor 400, but rather an unobstructed view of the ASIC 815, the printed circuit board, and the connectors 818 attached to the printed circuit board is provided when observing the pressure sensor 814 from the top side shown in FIG. 8A.

As shown in FIGS. 8A and 8C, one or more electrical connectors 818 (e.g., similar to electrical connector 720 of FIG. 7A) can extend from the printed circuit board of the pressure sensor 814, and one or more wires 816 can be connected between the connectors 818 of the pressure sensor 814 and the electronic control system 804. In some implementations, the wires 816 can be flexible wires, and in some implementations that include multiple wires 816, the wires can be grouped or bound together in a flexible ribbon cable. In contrast to the pressure sensor 400, the connectors 818 need not extend far from the printed circuit board (e.g., the connectors may extend less than 10 mm from the printed circuit board), and one or more flexible cables can electrically connect the connectors 818 to the electronic control system 804.

In FIGS. 8B and 8C, a second portion of the hermetic housing 830 is shown in addition to the system 800 that includes the manifold 802, the electronic control system 804, and the energy storage device 806 contained within the first portion of a hermetic housing 808. The manifold 802, the electronic control system 804, and the energy storage device 806 can be attached (e.g., adhesively bonded, welded, soldered, mechanically coupled, etc.) to the first portion of the hermitic housing 808, and then, after electrical connections are established between the electronic control system 804 and the pressure sensor 814 and between the electronic control system 804 and the fluid control devices 810, 812, the second portion of the hermetic housing 830 can be hermetically sealed to the first portion of the hermetic housing 808 to hermetically includes components 804, 806, 802, 810, 812, and 814 within an enclosed cavity defined by the portions 808, 830 of the hermetic housing. In some implementations, the first and second portions 808, 830 of the hermetic housing can include metal (e.g., titanium), and the second portion of the hermetic housing 830 can be hermetically sealed to the first portion of the hermetic housing 808 by welding the second portion to a top perimeter edge of the first portion. The height, h′, of the cavity must be sufficient for the components of the system 800 to fit within the enclosed cavity defined by the portions 808, 830 of the hermetic housing. Although the enclosed cavity is shown as being formed from a rectilinear-shaped first portion of the hermetic housing 808 and a flat second portion of the hermetic housing 830, other shapes of the different portions of the hermetic housing also are possible.

Referring again to FIGS. 7A and 7B, with the housing 702 of the pressure sensor 700 including a flange 708 having a diameter smaller than the diameter of the second circular well 524 of the recess or receptacle 520 in which the pressure sensor is seated, with the housing 702 of the pressure sensor 700 not having a diameter greater than the diameter of the second circular well 524, and with the flange 708 having a thickness that is less than a depth of the second circular well from a top surface 506 of the manifold in which the receptacle 520 is formed, the housing of the pressure sensor does not extend above the top surface 506 of the manifold, and the housing 702 of the pressure sensor 700 can be contained entirely within the receptacle. Furthermore, because the pressure sensor 700 does not include a top plate, the electrical connectors 720 of the pressure sensor can be contained entirely within the receptacle 520 (i.e., below the top surface 506 of the housing that defines the receptacle), and flexible wires can be connected to the connectors 720 within the receptacle and below the top surface of the housing. Because of this configuration of the pressure sensor 700 and this arrangement of the pressure sensor with respect to the manifold 500, the height, h′, of the cavity formed by housing portion 808 and housing portion 830 in which the system 800 fits can be less than the height, h, of the cavity formed by housing portion 608 and housing portion 630 in which the system 600 fits, which reduces the volume of a component of implantable device 200.

In addition to adapting the configuration of the pressure sensor for use within the hermetically-sealed cavity to reduce the volume of the enclosed cavity needed to accommodate the components of the system, components of the pressure sensor can be allocated between the manifold 802 and the electronic control system 804, so as so decrease, so as to reduce the height and volume of the manifold 802. For example, in some implementations, components of the pressure sensor can be located in the electronic control system 804 rather than in the manifold 802, so that the height or thickness of the manifold 802 can be correspondingly reduced. In some implementations, the ASIC 815 can be located on a PCBA of the electronic control system 804, rather than within the manifold 802. In some implementations, one or more components of the ASIC 815 (e.g., an amplifier, an analog-to-digital converter (ADC), a calibration or compensation circuit, etc. can be located in the electronic control system 804 rather than in the manifold 802, so the ASIC located in the manifold can be reduced in size, so that the height or thickness of the manifold 802 can be correspondingly reduced.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid operated device, comprising:
a fluid reservoir;
an inflatable member; and
a fluid control system configured to control fluid flow between the fluid reservoir and the inflatable member, the fluid control system including:
a manifold including a fluidic architecture defining one or more fluid passageways within in the manifold, and the manifold defining a plurality of receptacles, each receptacle configured for receiving a fluid control device;
at least one pump positioned in a first receptacle of the receptacles and in fluidic connection with at least one of the one or more fluid passageways, the at least one pump being configured to pump fluid from the fluid reservoir to the inflatable member;
a pressure sensor positioned in a second receptacle of the receptacles and in fluidic connection with one of the fluid passageways, the pressure sensor including:
a metal housing including one or more interior cavities, the metal housing being configured to fit entirely within the second receptacle;
electrical circuitry configured for converting a pressure into an electrical signal;
a flexible metal diaphragm attached to the metal housing and having a first portion positioned between an interior cavity of the one or more interior cavities and a fluid passageway and the first portion being configured to move inward and outward with respect to an interior cavity in response to a fluid pressure in the fluid passageway.

2. The implantable fluid operated device of claim 1, wherein the second receptacle includes a substantially circular shelf, and wherein the metal housing of the pressure sensor includes a substantially circular flange seated on the substantially circular shelf.

3. The implantable fluid operated device of claim 2, wherein a depth of the substantially circular shelf below a top surface of the manifold is greater than a thickness of the flange.

4. The implantable fluid operated device of claim 2, wherein the flange is located at an end of the metal housing.

5. The implantable fluid operated device of claim 1, wherein the manifold includes metal, and wherein the housing of the pressure sensor is welded to the manifold.

6. The implantable fluid operated device of claim 1, further comprising a hermetically-sealed housing that contains the fluid control system, an electronic control system, and an energy storage device.

7. The implantable fluid operated device of claim 6, wherein the hermetically-sealed housing includes a first portion configured for receiving the fluid control system, the electronic control system, and the energy storage device and a second portion that is hermetically-sealed to the first portion.

8. The implantable fluid operated device of claim 7, wherein the pressure sensor further includes a printed circuit board, the printed circuit board including a plurality of electrical connectors configured for providing electrical signals generated by the pressure sensor for transmission to the electronic control system.

9. The implantable fluid operated device of claim 8, wherein the pressure sensor includes a receptacle within the metal housing, wherein the receptacle is defined by an inner cylindrical wall of the metal housing and by the printed circuit board.

10. The implantable fluid operated device of claim 8, further comprising a plurality of flexible wires that electrically connect the plurality of electrical connectors to the electronic control system.

11. The implantable fluid operated device of claim 10, wherein the flexible wires are electrically connected to the plurality of electrical connectors within the receptacle within the metal housing.

12. The implantable fluid operated device of claim 6, wherein the electronic control system includes an ASIC configured for processing electrical signals received from a MEMS sensor of the pressure sensor and wherein the pressure sensor in the fluid control systems does not include an ASIC.

13. The implantable fluid operated device of claim 6, wherein the electronic control system includes an ADC configured for processing electrical signals received from a MEMS sensor of the pressure sensor and wherein the pressure sensor in the fluid control systems does not include an ADC.

14. The implantable fluid operated device of claim 1, wherein the pressure sensor does not extend out of the second receptacle above a top surface of the manifold.

15. A pressure sensor configured to be positioned in a receptacle of a manifold of a fluid control system and in fluidic connection with fluid in a fluid passageway defined by the manifold, the pressure sensor comprising:
a metal housing including one or more interior cavities, the metal housing being configured to fit entirely within the receptacle;
a flexible metal diaphragm attached to the metal housing and having a first portion positioned between an interior cavity of the one or more interior cavities and the fluid passageway and the first portion being configured to move inward and outward with respect to an interior cavity in response to a fluid pressure in the fluid passageway; and
electrical circuitry configured for converting a pressure of fluid in the fluid passageway into an electrical signal.

16. The pressure sensor of claim 15, wherein the metal housing of the pressure sensor includes a substantially circular flange configured to seat on a substantially circular shelf within the receptacle of the manifold.

17. The pressure sensor of claim 16, wherein a depth of the substantially circular shelf below a top surface of the manifold is greater than a thickness of the flange and wherein the flange is located at an end of the metal housing.

18. The pressure sensor of claim 15, wherein the metal housing includes titanium.

19. The pressure sensor of claim 15, wherein the pressure sensor in the fluid control systems does not include an ASIC configured for processing electrical signals received from a MEMS sensor of the pressure sensor.

20. The pressure sensor of claim 15, wherein the pressure sensor in the fluid control systems does not include an ADC configured for processing electrical signals received from a MEMS sensor of the pressure sensor.
